(19) United States
(12) Patent Application Publication
(10) Pub. No.: US 2025/0031766 A1
(43) Pub. Date: Jan. 30, 2025

(54) AEROSOL PROVISION SYSTEM

(71) Applicant: NICOVENTURES TRADING LIMITED, London (GB)

(72) Inventor: Joseph Peter SUTTON, London (GB)

(21) Appl. No.: 18/709,192

(22) PCT Filed: Oct. 24, 2022
(86) PCT No.: PCT/GB2022/052704

§ 371 Date: May 10, 2024

(30) Foreign Application Priority Data
Nov. 10, 2021 (GB) 2116166.6

(51) Int. Cl. A24F 40/51; A24F 40/53; A24F 40/57; A24F 40/60; G10L 17/22

(52) U.S. Cl. CPC A24F 40/51; A24F 40/53; A24F 40/57; A24F 40/60; G10L 17/22

(57) ABSTRACT

An aerosol provision system (1) for generating an aerosol, wherein the aerosol provision system (1) comprises a sensor (95), comprising a microphone (97), for detecting the voice of a user of the aerosol provision system (1). The aerosol provision system further comprises control circuitry (16) which is configured to receive sensor data from the sensor; determine from the sensor data whether the sensor data satisfies a predetermined criterion; and perform an operation of the aerosol provision system (1) in response to determining that both i) the sensor data satisfies the predetermined criterion and ii) an actuator 98 from the aerosol provision system (1) is being operated.

### FIELD

The present disclosure relates to aerosol provision systems such as, but not limited to, nicotine delivery systems (e.g. electronic cigarettes and the like).

### BACKGROUND

Electronic aerosol provision systems often employ an electronic cigarette (e-cigarette) or more generally an aerosol provision device. Such an aerosol provision system typically contains aerosolisable material (also called aerosol-generating material), such as a reservoir of fluid or liquid containing a formulation, typically but not necessarily including nicotine, or a solid material such as a tobacco-based product, from which a vapour/aerosol is generated for inhalation by a user, for example through heat vaporisation. Thus, an aerosol provision system will typically comprise a vaporiser (also called an aerosol generator), e.g. a heating element, arranged to aerosolise a portion of aerosolisable material to generate a vapour.

Once a vapour has been generated, the vapour may be passed through flavouring material to add flavour to the vapour (if the aerosolisable material was not itself flavoured), after which the (flavoured) vapour may be then delivered to a user via a mouthpiece from the aerosol provision system.

A potential drawback of a number of existing aerosol provision systems and associated aerosol provision devices is in respect of their inability to provide suitable operability to those with physical impediments, e.g. for those with a relative loss of hand co-ordination. Further drawbacks of a number of existing aerosol provision systems are that these can often be quite difficult to operate in low-light conditions. Various approaches are therefore described herein which seek to help address or mitigate some of these issues, through the use of aerosol provision systems which can be at least partially voice-controlled, and whose voice-controls can be customised, in advance of performing a particular (e.g. vaporising) operation of the aerosol provision system, as part of a separate setup/initial mode of operation. Also disclosed herein are voice-controlled aerosol provision systems which are configured to be at least partially resistant to inadvertent voice activation.

### SUMMARY

According to a first aspect of certain embodiments there is provided an aerosol provision system for generating an aerosol, wherein the aerosol provision system comprises: a sensor, comprising a microphone, for detecting the voice of a user of the aerosol provision system;
an actuator; and
control circuitry which is configured to:
   receive sensor data from the sensor;
   determine from the sensor data whether the sensor data satisfies a predetermined criterion; and
   perform an operation of the aerosol provision system only in response to determining that both i) the sensor data satisfies the predetermined criterion and ii) the actuator is being operated.

According to a second aspect of certain embodiments there is provided an aerosol provision device, for use in an aerosol provision system for generating an aerosol comprising the aerosol provision device and a consumable comprising aerosol-generating material for aerosolising, wherein the aerosol provision device comprises:
a sensor, comprising a microphone, for detecting the voice of a user of the aerosol provision device;
an actuator; and
control circuitry which is configured to:
   receive sensor data from the sensor;
   determine from the sensor data whether the sensor data satisfies a predetermined criterion; and
   perform an operation of the aerosol provision device only in response to determining that both i) the sensor data satisfies the predetermined criterion and ii) the actuator is being operated.

According to a third aspect of certain embodiments there is provided a method of performing an operation in an aerosol provision system, the method comprising control circuitry from the aerosol provision system:
receiving sensor data from a sensor, comprising a microphone, from the aerosol provision system, for detecting the voice of a user of the aerosol provision system;
determining from the sensor data whether the sensor data satisfies a predetermined criterion; and
performing an operation of the aerosol provision system only in response to determining that both i) the sensor data satisfies the predetermined criterion and ii) an actuator from the aerosol provision system is being operated.

According to a fourth aspect of certain embodiments there is provided an aerosol provision system for generating an aerosol, wherein the aerosol provision system comprises:
a sensor, comprising a microphone, for detecting the voice of a user of the aerosol provision system;
a user input device; and
control circuitry which is configured to:
   receive sensor data from the sensor;
   determine from the sensor data whether the sensor data satisfies a predetermined criterion; and
   perform an operation of the aerosol provision system in response to determining that either: i) the sensor data satisfies the predetermined criterion; and/or ii) the user input device from the aerosol provision system is being operated.

According to a fifth aspect of certain embodiments there is provided an aerosol provision system for generating an aerosol, wherein the aerosol provision system comprises:
a sensor, comprising a microphone, for detecting the voice of a user of the aerosol provision system;
an actuator; and
control circuitry which is configured to:
   receive sensor data from the sensor;
   determine from the sensor data whether the sensor data satisfies a predetermined criterion; and
   perform an operation of the aerosol provision system in response to determining that either: i) the sensor data satisfies the predetermined criterion; and/or ii) the actuator from the aerosol provision system is being operated.

According to a sixth aspect of certain embodiments there is provided a voice controlled aerosol provision system for generating an aerosol, wherein the aerosol provision system is configured to toggle between a first mode of operation in which the aerosol provision system is operable to be voice controlled for generating the aerosol, and a second mode of operation in which the aerosol provision system is not operable to be voice controlled for generating the aerosol.

According to a seventh aspect of certain embodiments there is provided a method of controlling the generation of aerosol in a voice controlled aerosol provision system, the method comprising switching the aerosol provision system between a first mode of operation in which the aerosol provision system is operable to be voice controlled for generating the aerosol, and a second mode of operation in which the aerosol provision system is not operable to be voice controlled for generating the aerosol.

It will be appreciated that features and aspects of the invention described above in relation to the various aspects of the invention are equally applicable to, and may be combined with, embodiments of the invention according to other aspects of the invention as appropriate, and not just in the specific combinations described herein.

### BRIEF DESCRIPTION OF THE DRAWINGS

Embodiments of the invention will now be described, by way of example only, with reference to the accompanying drawings, in which:

FIG. 1 schematically represents in perspective view an aerosol provision system comprising a cartridge and aerosol provision device (shown separated) in accordance with certain embodiments of the disclosure;

FIG. 2 schematically represents in exploded perspective view of components of the cartridge of the aerosol provision system of FIG. 1;

FIGS. 3A to 3C schematically represent various cross-section views of a housing part of the cartridge of the aerosol provision system of FIG. 1;

FIGS. 4A and 4B schematically represent a perspective view and a plan view of a dividing wall element of the cartridge of the aerosol provision system of FIG. 1;

FIGS. 5A to 5C schematically represent two perspective views and a plan view of a resilient plug of the cartridge of the aerosol provision system of FIG. 1;

FIGS. 6A and 6B schematically represent a perspective view and a plan view of a bottom cap of the cartridge of the aerosol provision system of FIG. 1;

FIG. 7 schematically represents embodiments of aerosol provision system, useable with an aerosol provision system such as that shown in FIGS. 1-6B, and which are configured to be at least partially voice-activated, in accordance with certain embodiments of the disclosure;

FIG. 8A schematically represents an embodiment of the aerosol provision system from FIG. 7, and which is configured to perform an operation of the aerosol provision system in response to determining that sensor data from a microphone of the aerosol provision system satisfies a predetermined criterion, which might be the sensor data being indicative of a predetermined utterance as having been spoken, and/or indicative of a voice pattern from a predetermined authorised user profile as having been spoken, in accordance with certain embodiments of the disclosure;

FIG. 8B schematically represents an embodiment of the aerosol provision system from FIG. 7, and which is configured to perform an operation of the aerosol provision system in response to determining that sensor data from a microphone of the aerosol provision system satisfies a second predetermined criterion, which might be the sensor data being indicative of a second predetermined utterance as having been spoken, in accordance with certain embodiments of the disclosure; and

FIG. 9 schematically represents an embodiment of the aerosol provision system from FIG. 7, and which further employs an actuator, in accordance with certain embodiments of the disclosure.

### DETAILED DESCRIPTION

Aspects and features of certain examples and embodiments are discussed/described herein. Some aspects and features of certain examples and embodiments may be implemented conventionally and these are not discussed/described in detail in the interests of brevity. It will thus be appreciated that aspects and features of apparatus and methods discussed herein which are not described in detail may be implemented in accordance with any conventional techniques for implementing such aspects and features.

The present disclosure relates to non-combustible aerosol provision systems (such as an e-cigarette). According to the present disclosure, a “non-combustible” aerosol provision system is one where a constituent aerosolisable material of the aerosol provision system (or component thereof) is not combusted or burned in order to facilitate delivery to a user. Aerosolisable material, which also may be referred to herein as aerosol generating material or aerosol precursor material, is material that is capable of generating aerosol, for example when heated, irradiated or energized in any other way. The aerosolisable material may also be flavoured, in some embodiments.

Throughout the following description the term “e-cigarette” or “electronic cigarette” may sometimes be used, but it will be appreciated this term may be used interchangeably with an aerosol provision system. An electronic cigarette may also known as a vaping device or electronic nicotine delivery system (END), although it is noted that the presence of nicotine in the aerosolisable material is not a requirement.

In some embodiments, the aerosol provision system is a hybrid device configured to generate aerosol using a combination of aerosolisable materials, one or a plurality of which may be heated. In some embodiments, the hybrid device comprises a liquid or gel aerosolisable material and a solid aerosolisable material. The solid aerosolisable material may comprise, for example, tobacco or a non-tobacco product.

Yet further still, in some embodiments, the aerosol provision system may be configured to generate aerosol using a solid aerosolisable material contained in a consumable type part. Here, the solid aerosolisable material may comprise, for example, tobacco or a non-tobacco product.

As noted above, the (non-combustible) aerosol provision system, in accordance with some embodiments, may comprise a cartridge/consumable part and a body/reusable/aerosol provision device part, which is configured to releasably engage with the cartridge/consumable part.

The aerosol provision system may be provided with a means for powering a vaporiser therein, and there may be provided an aerosolisable material transport element for receiving the aerosolisable material that is to be vaporised. The aerosol provision system may also be provided with a reservoir for containing aerosolisable material, and in some embodiments a further reservoir for containing flavouring material for flavouring a generated vapour from the aerosol provision system.

In some embodiments, the vaporiser may be a heater/heating element capable of interacting with the aerosolisable material so as to release one or more volatiles from the aerosolisable material to form a vapour/aerosol. In some embodiments, the vaporiser is capable of generating an aerosol from the aerosolisable material without heating. For example, the vaporiser may be capable of generating a vapour/aerosol from the aerosolisable material without applying heat thereto, for example via one or more of vibrational, mechanical, pressurisation or electrostatic means.

In some embodiments, the substance to be delivered may be an aerosolisable material which may comprise an active constituent, a carrier constituent and optionally one or more other functional constituents.

The active constituent may comprise one or more physiologically and/or olfactory active constituents which are included in the aerosolisable material in order to achieve a physiological and/or olfactory response in the user. The active constituent may for example be selected from nutraceuticals, nootropics, and psychoactives. The active constituent may be naturally occurring or synthetically obtained. The active constituent may comprise for example nicotine, caffeine, taurine, theine, a vitamin such as B6 or B12 or C, melatonin, a cannabinoid, or a constituent, derivative, or combinations thereof. The active constituent may comprise a constituent, derivative or extract of tobacco or of another botanical. In some embodiments, the active constituent is a physiologically active constituent and may be selected from nicotine, nicotine salts (e.g. nicotine ditartrate/nicotine bitartrate), nicotine-free tobacco substitutes, other alkaloids such as caffeine, or mixtures thereof.

In some embodiments, the active constituent is an olfactory active constituent and may be selected from a “flavour” and/or “flavourant” which, where local regulations permit, may be used to create a desired taste, aroma or other somatosensorial sensation in a product for adult consumers. In some instances such constituents may be referred to as flavours, flavourants, flavouring material, cooling agents, heating agents, and/or sweetening agents. They may include naturally occurring flavour materials, botanicals, extracts of botanicals, synthetically obtained materials, or combinations thereof (e.g., tobacco, cannabis, licorice (liquorice), hydrangea, eugenol, Japanese white bark magnolia leaf, chamomile, fenugreek, clove, maple, matcha, menthol, Japanese mint, aniseed (anise), cinnamon, turmeric, Indian spices, Asian spices, herb, wintergreen, cherry, berry, red berry, cranberry, peach, apple, orange, mango, clementine, lemon, lime, tropical fruit, papaya, rhubarb, grape, durian, dragon fruit, cucumber, blueberry, mulberry, citrus fruits, Drambuie, bourbon, scotch, whiskey, gin, tequila, rum, spearmint, peppermint, lavender, aloe vera, cardamom, celery, cascarilla, nutmeg, sandalwood, bergamot, geranium, khat, naswar, betel, shisha, pine, honey essence, rose oil, vanilla, lemon oil, orange oil, orange blossom, cherry blossom, cassia, caraway, cognac, jasmine, ylang-ylang, sage, fennel, wasabi, piment, ginger, coriander, coffee, hemp, a mint oil from any species of the genus Mentha, eucalyptus, star anise, cocoa, lemongrass, rooibos, flax, ginkgo biloba, hazel, hibiscus, laurel, mate, orange skin, rose, tea such as green tea or black tea, thyme, juniper, elderflower, basil, bay leaves, cumin, oregano, paprika, rosemary, saffron, lemon peel, mint, beefsteak plant, curcuma, cilantro, myrtle, cassis, valerian, pimento, mace, damien, marjoram, olive, lemon balm, lemon basil, chive, carvi, verbena, tarragon, limonene, thymol, camphene), flavour enhancers, bitterness receptor site blockers, sensorial receptor site activators or stimulators, sugars and/or sugar substitutes (e.g., sucralose, acesulfame potassium, aspartame, saccharine, cyclamates, lactose, sucrose, glucose, fructose, sorbitol, or mannitol), and other additives such as charcoal, chlorophyll, minerals, botanicals, or breath freshening agents. They may be imitation, synthetic or natural ingredients or blends thereof. They may be in any suitable form, for example, liquid such as an oil, solid such as a powder, or gasone or more of extracts (e.g., licorice, hydrangea, Japanese white bark magnolia leaf, chamomile, fenugreek, clove, menthol, Japanese mint, aniseed, cinnamon, herb, wintergreen, cherry, berry, peach, apple, Drambuie, bourbon, scotch, whiskey, spearmint, peppermint, lavender, cardamom, celery, cascarilla, nutmeg, sandalwood, bergamot, geranium, honey essence, rose oil, vanilla, lemon oil, orange oil, cassia, caraway, cognac, jasmine, ylang-ylang, sage, fennel, piment, ginger, anise, coriander, coffee, or a mint oil from any species of the genus Mentha), flavour enhancers, bitterness receptor site blockers, sensorial receptor site activators or stimulators, sugars and/or sugar substitutes (e.g., sucralose, acesulfame potassium, aspartame, saccharine, cyclamates, lactose, sucrose, glucose, fructose, sorbitol, or mannitol), and other additives such as charcoal, chlorophyll, minerals, botanicals, or breath freshening agents. They may be imitation, synthetic or natural ingredients or blends thereof. They may be in any suitable form, for example, oil, liquid, or powder.

In some embodiments, the flavouring material (flavour) may comprise menthol, spearmint and/or peppermint. In some embodiments, the flavour comprises flavour components of cucumber, blueberry, citrus fruits and/or redberry. In some embodiments, the flavour comprises eugenol. In some embodiments, the flavour comprises flavour components extracted from tobacco. In some embodiments, the flavour may comprise a sensate, which is intended to achieve a somatosensorial sensation which are usually chemically induced and perceived by the stimulation of the fifth cranial nerve (trigeminal nerve), in addition to or in place of aroma or taste nerves, and these may include agents providing heating, cooling, tingling, numbing effect. A suitable heat effect agent may be, but is not limited to, vanillyl ethyl ether and a suitable cooling agent may be, but not limited to eucalyptol, WS-3.

The carrier constituent may comprise one or more constituents capable of forming an aerosol. In some embodiments, the carrier constituent may comprise one or more of glycerine, glycerol, propylene glycol, diethylene glycol, triethylene glycol, tetraethylene glycol, 1,3-butylene glycol, erythritol, meso-Erythritol, ethyl vanillate, ethyl laurate, a diethyl suberate, triethyl citrate, triacetin, a diacetin mixture, benzyl benzoate, benzyl phenyl acetate, tributyrin, lauryl acetate, lauric acid, myristic acid, and propylene carbonate.

The one or more other functional constituents may comprise one or more of pH regulators, colouring agents, preservatives, binders, fillers, stabilizers, and/or antioxidants.

As noted above, aerosol provision systems (e-cigarettes) may often comprise a modular assembly including both a reusable part (body—or aerosol provision device) and a replaceable consumable (cartridge) part. Devices conforming to this type of two-part modular configuration may generally be referred to as two-part devices. It is also common for electronic cigarettes to have a generally elongate shape. For the sake of providing a concrete example, certain embodiments of the disclosure described herein may comprise this kind of generally elongate two-part device employing consumable parts. However, it will be appreciated the underlying principles described herein may equally be adopted for other electronic cigarette configurations, for example modular devices comprising more than two parts, as devices conforming to other overall shapes, for example based on so-called box-mod high performance devices that typically have a more boxy shape.

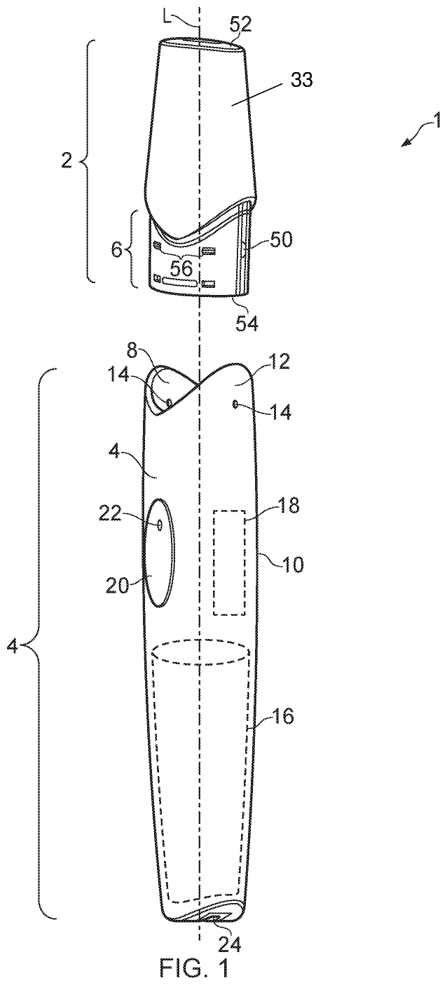

From the forgoing therefore, and with reference to FIG. 1 is a schematic perspective view of an example aerosol provision system (e-cigarette) 1 in accordance with certain embodiments of the disclosure. Terms concerning the relative location of various aspects of the electronic cigarette (e.g. terms such as upper, lower, above, below, top, bottom etc.) are used herein with reference to the orientation of the electronic cigarette as shown in FIG. 1 (unless the context indicates otherwise). However, it will be appreciated this is purely for ease of explanation and is not intended to indicate there is any required orientation for the electronic cigarette in use.

The e-cigarette 1 (aerosol provision system 1) comprises two main components, namely a cartridge 2 and an aerosol provision device 4. The aerosol provision device 4 and the cartridge 2 are shown separated in FIG. 1, but are coupled together when in use.

The cartridge 2 and aerosol provision device 4 are coupled by establishing a mechanical and electrical connection between them. The specific manner in which the mechanical and electrical connection is established is not of primary significance to the principles described herein and may be established in accordance with conventional techniques, for example based around a screw thread, bayonet, latched or friction-fit mechanical fixing with appropriately arranged electrical contacts/electrodes for establishing the electrical connection between the two parts as appropriate. For example electronic cigarette 1 represented in FIG. 1, the cartridge comprises a mouthpiece 33, a mouthpiece end 52 and an interface end 54 and is coupled to the aerosol provision device by inserting an interface end portion 6 at the interface end of the cartridge into a corresponding receptacle 8/receiving section of the aerosol provision device. The interface end portion 6 of the cartridge is a close fit to be receptacle 8 and includes protrusions 56 which engage with corresponding detents in the interior surface of a receptacle wall 12 defining the receptacle 8 to provide a releasable mechanical engagement between the cartridge and the aerosol provision device. An electrical connection is established between the aerosol provision device and the cartridge via a pair of electrical contacts on the bottom of the cartridge (not shown in FIG. 1) and corresponding sprung contact pins in the base of the receptacle 8 (not shown in FIG. 1). As noted above, the specific manner in which the electrical connection is established is not significant to the principles described herein, and indeed some implementations might not have an electrical connection between the cartridge and a aerosol provision device at all, for example because the transfer of electrical power from the reusable part to the cartridge may be wireless (e.g. based on electromagnetic induction techniques).

The electronic cigarette 1 (aerosol provision system) has a generally elongate shape extending along a longitudinal axis L. When the cartridge is coupled to the aerosol provision device, the overall length of the electronic cigarette in this example (along the longitudinal axis) is around 12.5 cm. The overall length of the aerosol provision device is around 9 cm and the overall length of the cartridge is around 5 cm (i.e. there is around 1.5 cm of overlap between the interface end portion 6 of the cartridge and the receptacle 8 of the aerosol provision device when they are coupled together). The electronic cigarette has a cross-section which is generally oval and which is largest around the middle of the electronic cigarette and tapers in a curved manner towards the ends. The cross-section around the middle of the electronic cigarette has a width of around 2.5 cm and a thickness of around 1.7 cm. The end of the cartridge has a width of around 2 cm and a thickness of around 0.6 mm, whereas the other end of the electronic cigarette has a width of around 2 cm and a thickness of around 1.2 cm. The outer housing of the electronic cigarette is in this example is formed from plastic. It will be appreciated the specific size and shape of the electronic cigarette and the material from which it is made is not of primary significance to the principles described herein and may be different in different implementations. That is to say, the principles described herein may equally be adopted for electronic cigarettes having different sizes, shapes and/or materials.

The aerosol provision device 4 may in accordance with certain embodiments of the disclosure be broadly conventional in terms of its functionality and general construction techniques. In the example of FIG. 1, the aerosol provision device 4 comprises a plastic outer housing 10 including the receptacle wall 12 that defines the receptacle 8 for receiving the end of the cartridge as noted above. The outer housing 10 of the aerosol provision device 4 in this example has a generally oval cross section conforming to the shape and size of the cartridge 2 at their interface to provide a smooth transition between the two parts. The receptacle 8 and the end portion 6 of the cartridge 2 are symmetric when rotated through 180° so the cartridge can be inserted into the aerosol provision device in two different orientations. The receptacle wall 12 includes two aerosol provision device air inlet openings 14 (i.e. holes in the wall). These openings 14 are positioned to align with an air inlet 50 for the cartridge when the cartridge is coupled to the aerosol provision device. A different one of the openings 14 aligns with the air inlet 50 of the cartridge in the different orientations. It will be appreciated some implementations may not have any degree of rotational symmetry such that the cartridge is couplable to the aerosol provision device in only one orientation while other implementations may have a higher degree of rotational symmetry such that the cartridge is couplable to the aerosol provision device in more orientations.

The aerosol provision device further comprises a battery 16 for providing operating power for the electronic cigarette, control circuitry 18 for controlling and monitoring the operation of the electronic cigarette, a user input button 20, an indicator light 22, and a charging port 24.

The battery 16 in this example is rechargeable and may be of a conventional type, for example of the kind normally used in electronic cigarettes and other applications requiring provision of relatively high currents over relatively short periods. The battery 16 may be recharged through the charging port 24, which may, for example, comprise a USB connector.

The input button 20 in this example is a conventional mechanical button, for example comprising a sprung mounted component which may be pressed by a user to establish an electrical contact in underlying circuitry. In this regard, the input button may be considered an input device for detecting user input, e.g. to trigger aerosol generation, and the specific manner in which the button is implemented is not significant. For example, other forms of mechanical button or touch-sensitive button (e.g. based on capacitive or optical sensing techniques) may be used in other implementations, or there may be no button and the device may rely on a puff detector for triggering aerosol generation.

The indicator light 22 is provided to give a user with a visual indication of various characteristics associated with the electronic cigarette, for example, an indication of an operating state (e.g. on/off/standby), and other characteristics, such as battery life or fault conditions. Different characteristics may, for example, be indicated through different colours and/or different flash sequences in accordance with generally conventional techniques.

The control circuitry 18 is suitably configured/programmed to control the operation of the electronic cigarette to provide conventional operating functions in line with the established techniques for controlling electronic cigarettes. The control circuitry (processor circuitry) 18 may be considered to logically comprise various sub-units/circuitry elements associated with different aspects of the electronic cigarette's operation. For example, depending on the functionality provided in different implementations, the control circuitry 18 may comprises power supply control circuitry for controlling the supply of power from the battery/power supply to the cartridge in response to user input, user programming circuitry for establishing configuration settings (e.g. user-defined power settings) in response to user input, as well as other functional units/circuitry associated functionality in accordance with the principles described herein and conventional operating aspects of electronic cigarettes, such as indicator light display driving circuitry and user input detection circuitry. It will be appreciated the functionality of the control circuitry 18 can be provided in various different ways, for example using one or more suitably programmed programmable computer(s) and/or one or more suitably configured application-specific integrated circuit(s)/circuitry/chip(s)/chipset(s) configured to provide the desired functionality.

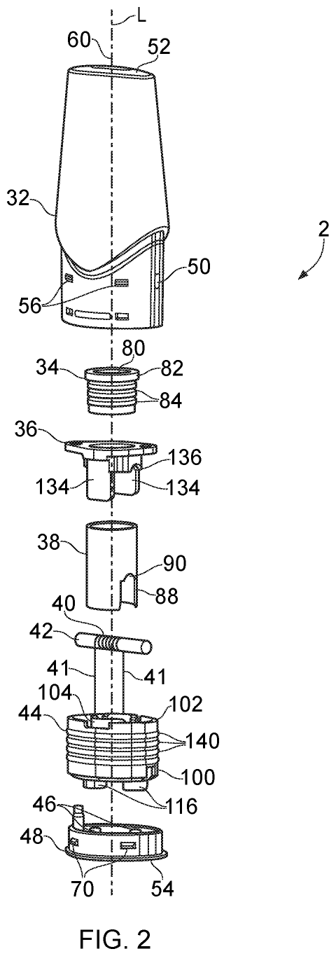

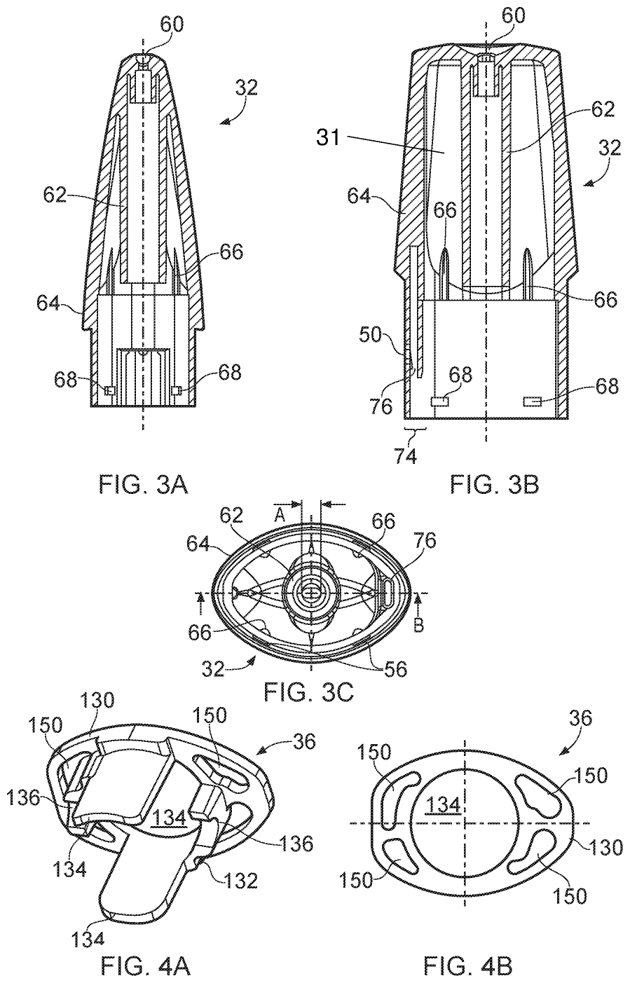

FIG. 2 is an exploded schematic perspective view of the cartridge 2 (exploded along the longitudinal axis L). The cartridge 2 comprises a housing part 32, an air channel seal 34, a dividing wall element 36, an outlet tube 38, a vaporiser/heating element 40, an aerosolisable material transport element 42, a plug 44, and an end cap 48 with contact electrodes 46. FIGS. 3 to 6 schematically represents some of these components in more detail.

FIG. 3A is a schematic cut-away view of the housing part 32 through the longitudinal axis L where the housing part 32 is thinnest. FIG. 3B is a schematic cut-away view of the housing part 32 through the longitudinal axis L where the housing part 32 is widest. FIG. 3C is a schematic view of the housing part along the longitudinal axis L from the interface end 54 (i.e. viewed from below in the orientation of FIGS. 3A and 3B).

FIG. 4A is a schematic perspective view of the dividing wall element 36 as seen from below. FIG. 4B is a schematic cross-section through an upper part of the dividing wall element 36 as viewed from below.

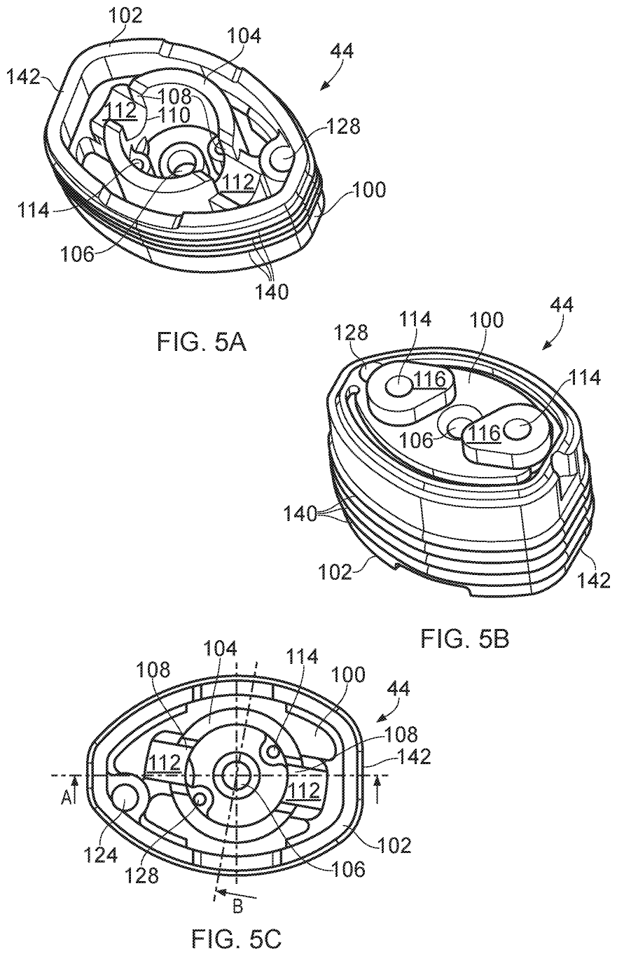

FIG. 5A is a schematic perspective view of the plug 44 from above and FIG. 5B is a schematic perspective view of the plug 44 from below. FIG. 5C is a schematic view of the plug 44 along the longitudinal axis L seen from the mouthpiece end 52 of the cartridge (i.e. viewed from above for the orientation in FIGS. 1 and 2).

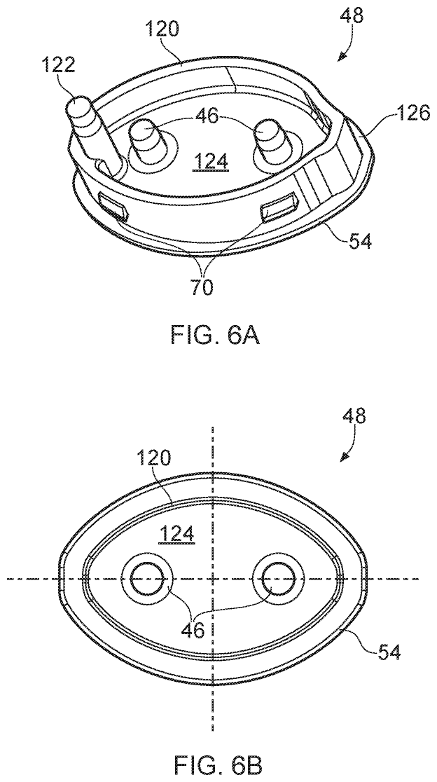

FIG. 6A is a schematic perspective view of the end cap 48 from above. FIG. 6B is a schematic view of the end cap 48 along the longitudinal axis L seen from the mouthpiece end 52 of the cartridge (i.e. from above).

The housing part 32 in this example comprises a housing outer wall 64 and a housing inner tube 62 which in this example are formed from a single moulding of polypropylene. The housing outer wall 64 defines the external appearance of the cartridge 2 and the housing inner tube 62 defines a part the air channel through the cartridge. The housing part is open at the interface end 54 of the cartridge and closed at the mouthpiece end 52 of the cartridge except for a mouthpiece opening/aerosol outlet 60, from the mouthpiece 33, which is in fluid communication with the housing inner tube 62. The housing part 32 includes an opening in a sidewall which provides the air inlet 50 for the cartridge. The air inlet 50 in this example has an area of around 2 mm². The outer surface of the outer wall 64 of the housing part 32 includes the protrusions 56 discussed above which engage with corresponding detents in the interior surface of the receptacle wall 12 defining the receptacle 8 to provide a releasable mechanical engagement between the cartridge and the aerosol provision device. The inner surface of the outer wall 64 of the housing part includes further protrusions 66 which act to provide an abutment stop for locating the dividing wall element 36 along the longitudinal axis L when the cartridge is assembled. The outer wall 64 of the housing part 32 further comprises holes which provide latch recesses 68 arranged to receive corresponding latch projections 70 in the end cap to fix the end cap to be housing part when the cartridge is assembled.

The outer wall 64 of the housing part 32 includes a double-walled section 74 that defines a gap 76 in fluid communication with the air inlet 50. The gap 76 provides a portion of the air channel through the cartridge. In this example the doubled-walled section 74 of the housing part 32 is arranged so the gap defines an air channel running within the housing outer wall 64 parallel to the longitudinal axis with a cross-section in a plane perpendicular to the longitudinal axis of around 3 mm². The gap/portion of air channel 76 defined by the double-walled section of the housing part extends down to the open end of the housing part 32.

The air channel seal 34 is a silicone moulding generally in the form of a tube having a through hole 80. The outer wall of the air channel seal 34 includes circumferential ridges 84 and an upper collar 82. The inner wall of the air channel seal 34 also includes circumferential ridges, but these are not visible in FIG. 2. When the cartridge is assembled the air channel seal 34 is mounted to the housing inner tube 62 with an end of the housing inner tube 62 extending partly into the through hole 80 of the air channel seal 34. The through hole 80 in the air channel seal has a diameter of around 5.8 mm in its relaxed state whereas the end of the housing inner tube 62 has a diameter of around 6.2 mm so that a seal is formed when the air channel seal 34 is stretched to accommodate the housing inner tube 62. This seal is facilitated by the ridges on the inner surface of the air channel seal 34.

The outlet tube 38 comprises a tubular section, for instance made of ANSI 304 stainless steel or polypropylene, with an internal diameter of around 8.6 mm and a wall thickness of around 0.2 mm. The bottom end of the outlet tube 38 includes a pair of diametrically opposing slots 88 with an end of each slot having a semi-circular recess 90. When the cartridge is assembled the outlet tube 38 mounts to the outer surface of the air channel seal 34. The outer diameter of the air channel seal is around 9.0 mm in its relaxed state so that a seal is formed when the air channel seal 34 is compressed to fit inside the outlet tube 38. This seal is facilitated by the ridges 84 on the outer surface of the air channel seal 34. The collar 80 on the air channel seal 34 provides a stop for the outlet tube 38.

The aerosolisable material transport element 42 comprises a capillary wick and the vaporiser (aerosol generator) 40 comprises a resistance wire heater wound around the capillary wick. In addition to the portion of the resistance wire wound around the capillary wick, the vaporiser comprises electrical leads 41 which pass through holes in the plug 44 to contact electrodes 46 mounted to the end cap 54 to allow power to be supplied to the vaporiser via the electrical interface the established when the cartridge is connected to an aerosol provision device. The vaporiser leads 41 may comprise the same material as the resistance wire wound around the capillary wick, or may comprise a different material (e.g. lower-resistance material) connected to the resistance wire wound around the capillary wick. In this example the heater coil 40 comprises a nickel iron alloy wire and the wick 42 comprises a glass fibre bundle. The vaporiser and aerosolisable material transport element may be provided in accordance with any conventional techniques and is may comprise different forms and/or different materials. For example, in some implementations the wick may comprise fibrous or solid a ceramic material and the heater may comprise a different alloy. In other examples the heater and wick may be combined, for example in the form of a porous and a resistive material. More generally, it will be appreciated the specific nature aerosolisable material transport element and vaporiser is not of primary significance to the principles described herein.

When the cartridge is assembled, the wick 42 is received in the semi-circular recesses 90 of the outlet tube 38 so that a central portion of the wick about which the heating coil is would is inside the outlet tube while end portions of the wick are outside the outlet tube 38.

The plug 44 in this example comprises a single moulding of silicone, may be resilient. The plug comprises a base part 100 with an outer wall 102 extending upwardly therefrom (i.e. towards the mouthpiece end of the cartridge). The plug further comprises an inner wall 104 extending upwardly from the base part 100 and surrounding a through hole 106 through the base part 100.

The outer wall 102 of the plug 44 conforms to an inner surface of the housing part 32 so that when the cartridge is assembled the plug in 44 forms a seal with the housing part 32. The inner wall 104 of the plug 44 conforms to an inner surface of the outlet tube 38 so that when the cartridge is assembled the plug 44 also forms a seal with the outlet tube 38. The inner wall 104 includes a pair of diametrically opposing slots 108 with the end of each slot having a semi-circular recess 110. Extended outwardly (i.e. in a direction away from the longitudinal axis of the cartridge) from the bottom of each slot in the inner wall 104 is a cradle section 112 shaped to receive a section of the aerosolisable material transport element 42 when the cartridge is assembled. The slots 108 and semi-circular recesses 110 provided by the inner wall of the plug 44 and the slots 88 and semi-circular recesses 90 of the outlet tube 38 are aligned so that the slots 88 in the outlet tube 38 accommodate respective ones of the cradles 112 with the respective semi-circular recesses in the outlet tube and plug cooperating to define holes through which the aerosolisable material transport element passes. The size of the holes provided by the semi-circular recesses through which the aerosolisable material transport element passes correspond closely to the size and shape of the aerosolisable material transport element, but are slightly smaller so a degree of compression is provided by the resilience of the plug 44. This allows aerosolisable material to be transported along the aerosolisable material transport element by capillary action while restricting the extent to which aerosolisable material which is not transported by capillary action can pass through the openings. As noted above, the plug 44 includes further openings 114 in the base part 100 through which the contact leads 41 for the vaporiser pass when the cartridge is assembled. The bottom of the base part of the plug includes spacers 116 which maintain an offset between the remaining surface of the bottom of the base part and the end cap 48. These spacers 116 include the openings 114 through which the electrical contact leads 41 for the vaporiser pass.

The end cap 48 comprises a polypropylene moulding with a pair of gold-plated copper electrode posts 46 mounted therein.

The ends of the electrode posts 44 on the bottom side of the end cap are close to flush with the interface end 54 of the cartridge provided by the end cap 48. These are the parts of the electrodes to which correspondingly aligned sprung contacts in the aerosol provision device 4 connect when the cartridge 2 is assembled and connected to the aerosol provision device 4. The ends of the electrode posts on the inside of the cartridge extend away from the end cap 48 and into the holes 114 in the plug 44 through which the contact leads 41 pass. The electrode posts are slightly oversized relative to the holes 114 and include a chamfer at their upper ends to facilitate insertion into the holes 114 in the plug where they are maintained in pressed contact with the contact leads for the vaporiser by virtue of the plug.

The end cap has a base section 124 and an upstanding wall 120 which conforms to the inner surface of the housing part 32. The upstanding wall 120 of the end cap 48 is inserted into the housing part 32 so the latch projections 70 engage with the latch recesses 68 in the housing part 32 to snap-fit the end cap 48 to the housing part when the cartridge is assembled. The top of the upstanding wall 120 of the end cap 48 abuts a peripheral part of the plug 44 and the lower face of the spacers 116 on the plug also abut the base section 124 of the plug so that when the end cap 48 is attached to the housing part it presses against the resilient part 44 to maintain it in slight compression.

The base portion 124 of the end cap 48 includes a peripheral lip 126 beyond the base of the upstanding wall 112 with a thickness which corresponds with the thickness of the outer wall of the housing part at the interface end of the cartridge. The end cap also includes an upstanding locating pin 122 which aligns with a corresponding locating hole 128 in the plug to help establish their relative location during assembly.

The dividing wall element 36 comprises a single moulding of polypropylene and includes a dividing wall 130 and a collar 132 formed by projections from the dividing wall 130 in the direction towards the interface end of the cartridge. The dividing wall element 36 has a central opening 134 through which the outlet tube 38 passes (i.e. the dividing wall is arranged around the outlet tube 38). In some embodiments, the dividing wall element 36 may be integrally formed with the outlet tube 38. When the cartridge is assembled, the upper surface of the outer wall 102 of the plug 44 engages with the lower surface of the dividing wall 130, and the upper surface of the dividing wall 130 in turn engages with the projections 66 on the inner surface of the outer wall 64 of the housing part 32. Thus, the dividing wall 130 prevents the plug from being pushed too far into the housing part 32—i.e. the dividing wall 130 is fixedly located along the longitudinal axis of the cartridge by the protrusions 66 in the housing part and so provides the plug with a fixed surface to push against. The collar 132 formed by projections from the dividing wall includes a first pair of opposing projections/tongues 134 which engage with corresponding recesses on an inner surface of the outer wall 102 of the plug 44. The protrusions from the dividing wall 130 further provide a pair of cradle sections 136 configured to engage with corresponding ones of the cradle sections 112 in the part 44 when the cartridge is assembled to further define the opening through which the aerosolisable material transport element passes.

When the cartridge 2 is assembled an air channel extending from the air inlet 50 to the aerosol outlet 60 through the cartridge is formed. Starting from the air inlet 50 in the side wall of the housing part 32, a first section of the air channel is provided by the gap 76 formed by the double-walled section 74 in the outer wall 64 of the housing part 32 and extends from the air inlet 50 towards the interface end 54 of the cartridge and past the plug 44. A second portion of the air channel is provided by the gap between the base of the plug 44 and the end cap 48. A third portion of the air channel is provided by the hole 106 through the plug 44. A fourth portion of the air channel is provided by the region within the inner wall 104 of the plug and the outlet tube around the vaporiser 40. This fourth portion of the air channel may also be referred to as an aerosol/aerosol generation region, it being the primary region in which aerosol is generated during use. The air channel from the air inlet 50 to the aerosol generation region may be referred to as an air inlet section of the air channel. A fifth portion of the air channel is provided by the remainder of the outlet tube 38. A sixth portion of the air channel is provided by the outer housing inner tube 62 which connects the air channel to the aerosol outlet 60, which is located at an end of the mouthpiece 33. The air channel from the aerosol generation region to be the aerosol outlet may be referred to as an aerosol outlet section of the air channel.

Also, when the cartridge is assembled a reservoir 31 for aerosolisable material is formed by the space outside the air channel and inside the housing part 32. This may be filled during manufacture, for example through a filling hole which is then sealed, or by other means. The specific nature of the aerosolisable material, for example in terms of its composition, is not of primary significance to the principles described herein, and in general any conventional aerosolisable material of the type normally used in electronic cigarettes may be used. The present disclosure may refer to a liquid as the aerosolisable material, which as mentioned above may be a conventional e-liquid. However, the principles of the present disclosure apply to any aerosolisable material which has the ability to flow, and may include a liquid, a gel, or a solid, where for a solid a plurality of solid particles may be considered to have the ability to flow when considered as a bulk.

The reservoir is closed at the interface end of the cartridge by the plug 44. The reservoir includes a first region above the dividing wall 130 and a second region below the dividing wall 130 within the space formed between the air channel and the outer wall of the plug. The aerosolisable material transport element (capillary wick) 42 passes through openings in the wall of the air channel provided by the semi-circular recesses 108, 90 in the plug 44 and the outlet tube 38 and the cradle sections 112, 136 in the plug 44 and the dividing wall element 36 that engage with one another as discussed above. Thus, the ends of the aerosolisable material transport element extend into the second region of the reservoir from which they draw aerosolisable material through the openings in the air channel to the vaporiser 40 for subsequent vaporisation.

In normal use, the cartridge 2 is coupled to the aerosol provision device 4 and the aerosol provision device activated to supply power to the cartridge via the contact electrodes 46 in the end cap 48. Power then passes through the connection leads 41 to the vaporiser 40. The vaporiser is thus electrically heated and so vaporises a portion of the aerosolisable material from the aerosolisable material transport element in the vicinity of the vaporiser. This generates aerosol in the aerosol generation region of the air path. Aerosolisable material that is vaporised from the aerosolisable material transport element is replaced by more aerosolisable material drawn from the reservoir by capillary action. While the vaporiser is activated, a user inhales on the mouthpiece end 52 of the cartridge. This causes air to be drawn through whichever aerosol provision device air inlet 14 aligns with the air inlet 50 of the cartridge (which will depend on the orientation in which the cartridge was inserted into the aerosol provision device receptacle 8). Air then enters the cartridge through the air inlet 50, passes along the gap 76 in the double-walled section 74 of the housing part 32, passes between the plug 44 and the end cap 48 before entering the aerosol generation region surrounding the vaporiser 40 through the hole 106 in the base part 100 of the plug 44. The incoming air mixes with aerosol generated from the vaporiser to form a condensation aerosol, which is then drawn along the outlet tube 38 and the housing part inner 62 before exiting through the mouthpiece outlet/aerosol outlet 60 for user inhalation.

From the above FIGS. 1-6B, it can be seen a possible embodiment construction of aerosol provision system 1 which is configured for generating an aerosol, which is suitable for use in the context of the present disclosure (alongside potentially other forms of aerosol provision system).

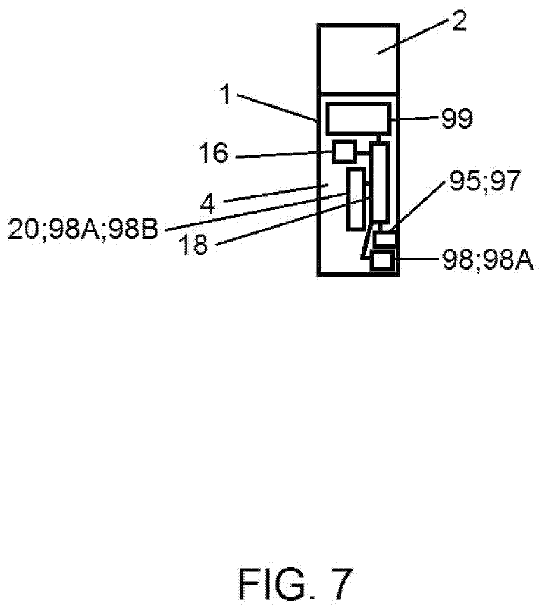

Turning now to FIGS. 7-9, in accordance with some embodiments, the present disclosure also provides for aerosol provision systems (which could be based off the aerosol provision system 1 as shown in FIGS. 1-6B, for instance-although other forms of aerosol provisions system could appreciably be also used, in so far as they are able to generate an aerosol) which are configured to be at least partially voice controlled.

At a general level therefore, herein provided in accordance with some embodiments of the disclosure may be a (voice controlled) aerosol provision system for generating an aerosol, wherein the aerosol provision system comprises a sensor 95, comprising a microphone 97, for detecting the voice of a user of the aerosol provision system 1. In this way, the aerosol provision system 1 may then comprise control circuitry 18 which is configured to receive sensor data from the sensor 95, determine from the sensor data whether the sensor data satisfies a predetermined criterion; and then perform an operation of the aerosol provision system 1 in response to determining that the sensor data satisfies the predetermined criterion.

By virtue of this arrangement therefore, the aerosol provision system 1 can be configured to allow an operation of the aerosol provision system 1, which could purely for example comprise an operation of providing power to the aerosol generator 40 from the aerosol provision system 1, and/or an operation comprising generating aerosol from the aerosol provision system 1, in response to an appropriate voice-command or utterance from the user of the aerosol provision system 1.

In accordance with some embodiments, the aerosol provision system 1 may be configured to operate in a mode of operation, prior to performing the operation of the aerosol provision system, for allowing the predetermined criterion to be specified. Such a mode of operation may be referred to as an ‘initial’ mode of operation or a ‘setup’ mode of operation, in accordance with some embodiments, which the user of the aerosol provision system 1 may access, to allow the user to more effectively tailor the predetermined criterion to them and their voice patterns, as will be described.

In this way therefore, through the presence of this initial/setup mode of operation, the aerosol provision system 1 may be better customised for only operating in response to commands or voice patterns of the particular intended user of the aerosol provision system, as opposed to inadvertently operating as a result of an utterance or voice pattern by someone else nearby the aerosol provision system 1.

Mindful of the above therefore, with respect to the above recited predetermined criterion, the predetermined criterion may in accordance with some embodiments comprise the sensor data being indicative of a predetermined utterance as having been spoken. By the term utterance as used herein, this may be understood as meaning a phrase, such as a word; sentence or some other vocal statement, which is uttered by the user of the aerosol provision system 1. In this way therefore, and in so far as the predetermined criterion comprises the sensor data being indicative of a predetermined utterance as having been spoken, the predetermined utterance in accordance could be indicative of a predetermined phrase as having been spoken.

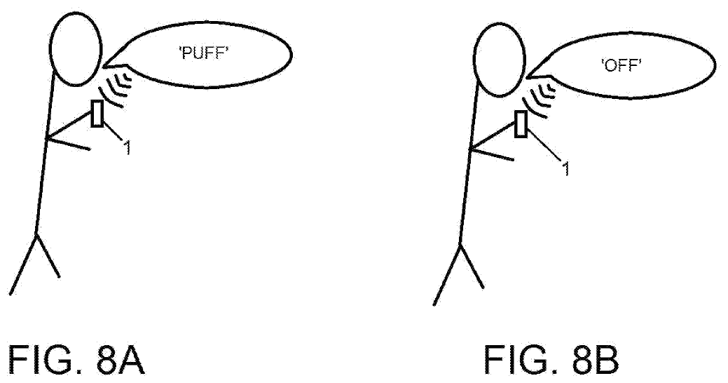

As to what such a predetermined utterance might be, it will be appreciated that this could be any utterance which is programmed into the control circuitry 18 of the aerosol provision system 1, such as an utterance of the phrase ‘puff’ (as shown in the embodiment of FIGS. 8A) ‘on’; ‘off’ (as shown in the embodiment of FIG. 8B); ‘more power’; ‘less power’; or some other utterance which is pre-specified by the user, such as ‘my voice is my password’.

Noting the above, it is to be appreciated that the aerosol provisions system 1 may be configured to allow the user to specify what the predetermined utterance should be for any given operation of the aerosol provision system which may be configured to be voice activated. In this way therefore, and in accordance with some embodiments, the aerosol provision system 1 may be configured to operate in the mode of operation for allowing the predetermined utterance to be specified, wherein the control circuitry 18 is configured, whilst the aerosol provision system 1 is operating in the mode of operation, to output a signal for instructing a user of the aerosol provision system 1 to specify an utterance. In this way, once the user has specified the utterance, the control circuitry 18 may then receive utterance data, from the sensor 95 (and/or the microphone 97) containing data, such as audio data, relating to the utterance.

Once the data is received from the sensor 95, the control circuitry 18 may then use voice recognition software, or speech-to-text software, which is stored in a memory of the aerosol provision system 1, to determine the utterance that was spoken by the user from the utterance data, and then from this specify the predetermined utterance as being the determined utterance.

By way of a series of examples therefore, to illustrate this functionality, it may be that in accordance with a particular embodiment, the user may wish to specify the predetermined criterion responsible for controlling when the aerosol provision system 1 operates the aerosol generator 40. In this way therefore, the user may use the initial/setup mode of operation to assign a predetermined utterance, of ‘puff’, to this predetermined criterion/operation. In this way, and through the user operating the aerosol provision system 1 in the mode of operation, in response to the control circuitry 18 outputting the signal for instructing the user to specify an utterance, the user may then speak the utterance ‘puff’. In this way, for operating the aerosol generator 40 of the aerosol provision system 1 moving forward, or until such time as a different utterance is assigned to the operation of the aerosol generator 40 of the aerosol provision system 1 (by repeating the mode of operation again for this particular operation), the user may then simply need to speak the utterance ‘puff’ near to the aerosol provision system 1.

With reference to the above functionality of the aerosol provision system 1, it may be appreciated that this ability to specify a particular utterance from the user to a particular operation of the aerosol provision system may be applicable to any required operation or operations of the aerosol provision system.

In this way therefore, and in accordance with some embodiments where multiple operations of the aerosol provision system are configured to be voice controlled, the control circuitry 18 may be configured, whilst the aerosol provision system 1 is operating in the mode of operation, to systemically instruct the user to specify a particular/respective utterance for each operation of the aerosol provision system which is configured to be voice controlled. In this way therefore, and at a general level, the present disclosure may therefore also provide for an aerosol provision system for generating an aerosol, wherein the aerosol provision system comprises the sensor, comprising the microphone, for detecting the voice of a user of the aerosol provision system, and wherein the aerosol provision system further comprises control circuitry which is configured to: receive sensor data from the sensor; determine from the sensor data whether the sensor data satisfies one of a plurality of predetermined criterions; and perform an operation of the aerosol provision system, assigned to the one of the plurality of predetermined criterions, in response to determining that the sensor data satisfies the predetermined criterion from the plurality of predetermined criterions.

In this way as well therefore, in accordance with such embodiments, the aerosol provision system may in some narrower embodiments be configured to operate in the mode of operation, prior to performing the operation of the aerosol provision system, for allowing each of the predetermined criterions [for each of the respective different operations of the aerosol provision system] to be specified.

As to how these (multiple) predetermined criterions may be specified, it is envisaged that the aerosol provision system may be configured to allow each of the predetermined criterions to be sequentially specified in some cases as part of the mode of operation. For instance, this may be achieved through the mode of operation carrying out the necessary user interaction steps for specifying a particular utterance for a first operation from the aerosol provision system, and then repeating these user interaction steps as part of the mode of operation for the remaining operations from the aerosol provision system whose operation is configured to be voice controlled.

Alternatively, in accordance with some embodiments, rather than cycling through each of the predetermined criterions as part of the mode of operation, in accordance with some embodiments, the aerosol provisions system 1 may comprise a user input device, such as the input button 20, or a touch-sensitive display 99 from the aerosol provision system 1, for allowing the or each operation of the aerosol provision system 1 to be specified. In this way, prior to, and/or as part of the mode of operation, the aerosol provision system 1 in accordance with some particular embodiments may be configured to allow the user to select, using the user input device 20;99, which predetermined criterion is to be specified in the mode of operation. In this way, the user of the aerosol provision system 1 then does not need to necessarily cycle through, or specify, all of the predetermined criterions [for all of the respective different operations of the aerosol provision system] as part of the mode of operation, if all they wish to do is to specify/change only one predetermined criterion for a particular operation of the aerosol provision system 1.

For completeness as well, and in so far as any user input device is employed, it may be appreciated that this user input device may similarly be used for allowing the predetermined criterion, such as any predetermined utterance or predetermined phrase therefrom, to be inputted by the user. In this way, rather than needing to recite the particular predetermined utterance or predetermined phrase as part of the mode of operation, the user may instead simply type/insert the particular predetermined utterance or predetermined phrase (e.g. the word ‘puff’) into the user input device 99. In this way, and when the sensor data from the sensor 95 is then later on determined by the control circuitry 18 as being indicative of this predetermined utterance (e.g. the word ‘puff’) as having been spoken, the control circuitry 18 may then perform the operation which is associated with this predetermined utterance.

Yet further still, and in so far as any user input device is employed, it may be appreciated that this user input device may similarly be used for allowing the operation of the aerosol provision system for any given predetermined criterion to be specified via the user input device. In this way, before, or as part of the mode of operation, the user input device may be employed to select which operation of the aerosol provision system (e.g. an operation to turn the aerosol provision system on, or a different operation to operate the aerosol generator 40 from the aerosol provision system, or yet another operation to turn the aerosol provision system 1 off) is to be related/paired to a particular predetermined criterion which is to be specified as part of the mode of operation.

Equally however, and in accordance with some embodiments, rather than needing to use the user input device to select which operation of the aerosol provision system 1 is to be related/paired to a particular predetermined criterion which is to be specified as part of the mode of operation, in accordance with some embodiments, the aerosol provision system may be configured for allowing the operation of the aerosol provision system to be specified via the sensor 95 or microphone 97—through the user verbally specifying/reciting the mode of operation whose predetermined criterion is to be specified.

From the foregoing therefore, it may be seen that the mode of operation from the aerosol provision system 1 may be employed to allow any/each given predetermined criterion, relating to any given operation of the aerosol provision system, to be specified to a particular utterance which the user so specifies as part of the mode of operation, to thus reduce the likelihood of each such operation of the aerosol provision system being inadvertently voice activated by a person who is not the user of the aerosol provision system 1.

With this mind, and in accordance with some related embodiments, any given predetermined criterion may comprise the sensor data being indicative of a voice pattern from a predetermined authorised user profile. In this way, the aerosol provision system 1 may be configured to require the user's voice pattern to be recognised, as opposed to necessarily only requiring that the user specifies a particular utterance (such as a particular word or phrase) for the control circuitry to perform the related operation from the aerosol provision system 1.

Where such voice pattern embodiments are employed, the mode of operation may again be configured such that the control circuitry 18 in this mode of operation outputs a signal for instructing a user of the aerosol provision system to specify an utterance, which results in the control circuitry receiving utterance data, from the sensor 95, containing data relating to the utterance. In these latter embodiments however, rather than necessarily being configured to determine an utterance (e.g. the word ‘puff’) from the utterance data, the control circuitry may additionally/alternatively be configured to determine a voice pattern from the utterance data; and then specify the voice pattern as being the voice pattern from the predetermined authorised user profile.

In these latter embodiments therefore, and by using voice recognition/profiling software (such as the voice profiling software employed on the Alexa® system owned by Amazon®) which is stored in the memory of the aerosol provision system 1, the control circuitry 18 may be configured to only allow the operation of the aerosol provision system 1 when a particular voice pattern is determined from the utterance data. So for instance, where a voice pattern of ‘Peter’ is specified as the voice pattern from the predetermined authorised user profile, as part of the mode of operation, if a different user (e.g. ‘Nigel’ or ‘Tracey’) then tries to operate the aerosol provision system 1 later on, the different voice pattern for this different user (‘Nigel’ or ‘Tracey’) will not match the voice pattern from the predetermined authorised user profile (namely the user profile relating to ‘Peter’). Accordingly the control circuitry 18 will not perform the operation of the aerosol provision system 1 for the different user (‘Nigel’ or ‘Tracey’). In this way therefore, the control circuitry 18 may thus limit the functionality of the aerosol provision system to only an authorised user (or users where more than predetermined authorised user profile is employed).

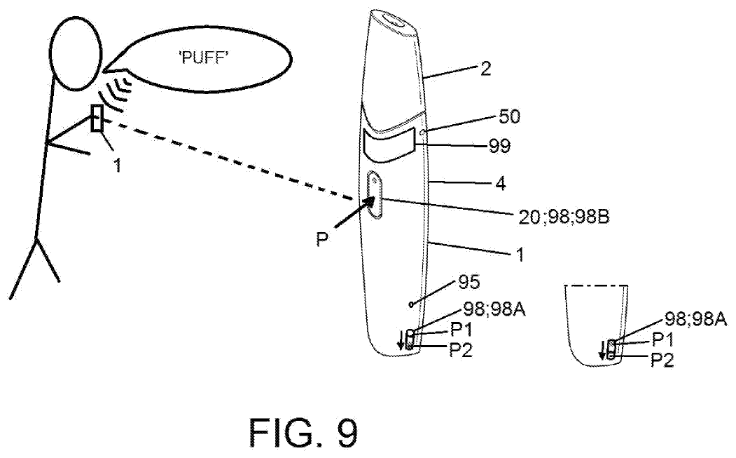

To help further inhibit any inadvertent voice-activation of the aerosol provision systems 1 herein described, in accordance with some embodiments, there may be required a two-factor input process for allowing any given operation of the aerosol provision system to be performed, as shown best with respect to the particular embodiments illustrated in FIG. 9. Thus in accordance with some embodiments, the control circuitry 18 from the aerosol provision system 1 may be configured to only perform the operation to the aerosol provision system 1 in response to determining that both i) the sensor data satisfies the predetermined criterion; and ii) an actuator 98 from the aerosol provision system 1 is being operated. In this way, and in so far as the user may accidentally recite a particular utterance relating to this predetermined criterion (e.g. the word ‘puff’ with reference to some of the earlier described embodiments), such as due to this utterance coming up as part of some other conversation which the user is having, reciting this utterance alone will then not cause the control circuitry 18 to perform the operation to the aerosol provision system 1.

As to what such an actuator 98 from the aerosol provision system 1 might be, it will be appreciated that this could notionally comprise a number of different things. For instance, and in accordance with some embodiments, this actuator 98 may comprise a touch sensor, or a capacitive sensor. Equally, and in accordance with some embodiments, the actuator 98 may be moveable between a first position in which the actuator is not operated, and a second position in which the actuator is operated. In this way, and in certain embodiments, the actuator may be toggled between the first position and the second position (e.g. as part of a slideable switch). Yet further still, and in accordance with some embodiments—such as that shown in the embodiment in FIG. 9, the actuator 98 could conveniently comprise the user input device, such as the user input button 20 and/or the touch-sensitive display 99. With reference to these embodiments therefore where the control circuitry 18 from the aerosol provision system 1 is configured to only perform the operation to the aerosol provision system in response to determining that both i) the sensor data satisfies the predetermined criterion; and ii) the actuator 20;98B from the aerosol provision system 1 is being operated, to yet further control the operation of the aerosol provision system 1 in these instances, in accordance with some embodiments (such as, for instance, in some embodiments where the operation comprises providing power to the aerosol generator 40 from the aerosol provision system 1), the control circuitry 18 may be configured to stop the operation as soon as the control circuitry 18 determines that the actuator 20;98B is no longer being operated. This being the case, and in embodiments for instance where the actuator 20;98 is biased from a second position as noted above towards the first position as noted above, as soon as the user then releases their pressure (P) on the actuator 20;98B, the actuator 20;98B may thus move back to the first position, to thus cause the control circuitry 18 to stop the operation of the aerosol provision system (which could be, for instance, a particular operation to provide power to the aerosol generator 40 of the aerosol provision system 1).

Not withstanding the above particular embodiments where the actuator 98 is employed, it is to be appreciated that this actuator 98 could also be employed in other embodiments to allow for a mechanism 98A for allowing the sensor 95 to operate. In this way, and unless the actuator 98A is operated, this may thus disable the sensor 95 from being operated, and thus disable any voice activation capabilities in the aerosol provision system 1. In such embodiments, the aerosol provision system 1 might still then be configured to be manually activated, such as per the operations described with reference to the embodiments of FIGS. 1-6B, using the user input button 20, for instance. Thus in accordance with these embodiments, the presence of the actuator 98;98A may allow for the aerosol provision system to toggle between a first mode of operation (as per the position P1 of the actuator 98A in the embodiment from FIG. 9) in which the aerosol provision system 1 is operable to be voice controlled (using the senor 95), and a second mode of operation (as per the position P2 of the actuator 98A in the embodiment from FIG. 9) in which the aerosol provision system 1 is not operable to be voice controlled (and/or is only operable to generate an aerosol in response to depressing the user input button 20). Such functionality may be particularly helpful, for instance, in instances where the aerosol provision system 1 is intended to be operated in an area where silence is required, and/or conversely in instances where there is too much ambient noise (such as in a concert) for the control circuitry 18 to be able to discern from the sensor data any particular utterances made by the user over this ambient noise.

Not withstanding the above embodiments where the actuator is employed 98, it is to be appreciated that the actuator 98 could additionally/alternatively be employed in other embodiments to allow for a different/separate mechanism for allowing the sensor 95 to operate, and/or for instructing the control circuitry 18 to perform the operation of the aerosol provision system 1 and/or. For instance, the actuator 98;98B may be configured to be operated for allowing the operation to be performed. In this way, the user may then have two separate mechanisms for performing the operation of the aerosol provision system, namely to either manually operate the actuator (which could be the user input button 20;98B, for instance) and/or through the use of the sensor 95 and the herein described voice activation mechanisms for the aerosol provision system 1. Thus again, in accordance with these embodiments, the presence of the actuator 98 may provide enhanced operability for the aerosol provision system 1.

Appreciating the foregoing therefore, there has accordingly been described an aerosol provision system for generating an aerosol, wherein the aerosol provision system comprises:
a sensor, comprising a microphone, for detecting the voice of a user of the aerosol provision system;
an actuator; and
control circuitry which is configured to:
   receive sensor data from the sensor;
   determine from the sensor data whether the sensor data satisfies a predetermined criterion; and
   perform an operation of the aerosol provision system only in response to determining that both i) the sensor data satisfies the predetermined criterion and ii) the actuator is being operated.

There has also been described an aerosol provision device, for use in an aerosol provision system for generating an aerosol comprising the aerosol provision device and a consumable comprising aerosol-generating material for aerosolising, wherein the aerosol provision device comprises:
a sensor, comprising a microphone, for detecting the voice of a user of the aerosol provision device;
an actuator; and
control circuitry which is configured to:
   receive sensor data from the sensor;
   determine from the sensor data whether the sensor data satisfies a predetermined criterion; and
   perform an operation of the aerosol provision device only in response to determining that both i) the sensor data satisfies the predetermined criterion and ii) the actuator is being operated.

There has also been described a method of performing an operation in an aerosol provision system, the method comprising control circuitry from the aerosol provision system:
receiving sensor data from a sensor, comprising a microphone, from the aerosol provision system, for detecting the voice of a user of the aerosol provision system;
determining from the sensor data whether the sensor data satisfies a predetermined criterion; and
performing an operation of the aerosol provision system only in response to determining that both i) the sensor data satisfies the predetermined criterion and ii) an actuator from the aerosol provision system is being operated.

There has also been described an aerosol provision system for generating an aerosol, wherein the aerosol provision system comprises:
a sensor, comprising a microphone, for detecting the voice of a user of the aerosol provision system;
a user input device; and
control circuitry which is configured to:
   receive sensor data from the sensor;
   determine from the sensor data whether the sensor data satisfies a predetermined criterion; and
   perform an operation of the aerosol provision system in response to determining that either: i) the sensor data satisfies the predetermined criterion; and/or ii) the user input device from the aerosol provision system is being operated.

There has also been described an aerosol provision system for generating an aerosol, wherein the aerosol provision system comprises:
a sensor, comprising a microphone, for detecting the voice of a user of the aerosol provision system;
an actuator; and
control circuitry which is configured to:
   receive sensor data from the sensor;
   determine from the sensor data whether the sensor data satisfies a predetermined criterion; and
   perform an operation of the aerosol provision system in response to determining that either: i) the sensor data satisfies the predetermined criterion; and/or ii) the actuator from the aerosol provision system is being operated.

There has also been described a voice controlled aerosol provision system for generating an aerosol, wherein the aerosol provision system is configured to toggle between a first mode of operation in which the aerosol provision system is operable to be voice controlled for generating the aerosol, and a second mode of operation in which the aerosol provision system is not operable to be voice controlled for generating the aerosol.

There has also been described a method of controlling the generation of aerosol in a voice controlled aerosol provision system, the method comprising switching the aerosol provision system between a first mode of operation in which the aerosol provision system is operable to be voice controlled for generating the aerosol, and a second mode of operation in which the aerosol provision system is not operable to be voice controlled for generating the aerosol.

There has also been described an aerosol provision system 1 for generating an aerosol, wherein the aerosol provision system 1 comprises a sensor 95, comprising a microphone 97, for detecting the voice of a user of the aerosol provision system 1. The aerosol provision system further comprises control circuitry 16 which is configured to receive sensor data from the sensor; determine from the sensor data whether the sensor data satisfies a predetermined criterion; and perform an operation of the aerosol provision system 1 in response to determining that both i) the sensor data satisfies the predetermined criterion and ii) an actuator 98 from the aerosol provision system 1 is being operated.

Also provided herein are the embodiments as specified in the following recited clauses:
1. An aerosol provision system for generating an aerosol, wherein the aerosol provision system comprises a sensor, comprising a microphone, for detecting the voice of a user of the aerosol provision system, and wherein the aerosol provision system further comprises control circuitry which is configured to:
   receive sensor data from the sensor;
   determine from the sensor data whether the sensor data satisfies a predetermined criterion; and
   perform an operation of the aerosol provision system in response to determining that the sensor data satisfies the predetermined criterion;
   wherein the aerosol provision system is configured to operate in a mode of operation, prior to performing the operation of the aerosol provision system, for allowing the predetermined criterion to be specified.
2. An aerosol provision system according to any preceding clause, wherein the predetermined criterion comprises the sensor data being indicative of a predetermined utterance as having been spoken.
3. An aerosol provision system according to clause 2, wherein the predetermined utterance is indicative of a predetermined phrase as having been spoken.
4. An aerosol provision system according to clause 2 or 3, wherein the aerosol provision system is further configured to operate in the mode of operation for allowing the predetermined utterance to be specified, wherein the control circuitry is configured, whilst the aerosol provision system is operating in the mode of operation, to:
   output a signal for instructing a user of the aerosol provision system to specify an utterance;
   receive utterance data, from the sensor, containing data relating to the utterance;
   determine the utterance from the utterance data; and
   specify the predetermined utterance as being the determined utterance.
5. An aerosol provision system according to any preceding clause, wherein the predetermined criterion comprises the sensor data being indicative of a voice pattern from a predetermined authorised user profile.
6. An aerosol provision system according to clause 5, wherein the aerosol provision system is further configured to operate in the mode of operation for allowing the voice pattern from the predetermined authorised user profile to be specified, wherein the control circuitry is configured, whilst the aerosol provision system is operating in the mode of operation, to:
   output a signal for instructing a user of the aerosol provision system to specify an utterance;
   receive utterance data, from the sensor, containing data relating to the utterance;
   determine the voice pattern from the utterance data; and
   specify the voice pattern as being the voice pattern from the predetermined authorised user profile.
7. An aerosol provision system according to any preceding clause, when further dependent on clause 4 or 6 at least, wherein the aerosol provision system is further configured for allowing the utterance to be specified as a predetermined phrase.
8. An aerosol provision system according to clause 7, wherein the aerosol provision system comprises a user input device for allowing the predetermined phrase to be inputted.
9. An aerosol provision system according to clause 8, wherein the user input device comprises a touch-sensitive display.
10. An aerosol provision system according to any preceding clause, wherein the aerosol provision system is further configured, in the mode of operation, for allowing the operation of the aerosol provision system, which is operated in response to determining that the sensor data satisfies the predetermined criterion, to be specified.
11. An aerosol provision system according to clause 10, wherein the aerosol provision system is configured for allowing the operation of the aerosol provision system to be specified via a user input device.
12. An aerosol provision system according to clause 10, wherein the aerosol provision system is configured for allowing the operation of the aerosol provision system to be specified via the microphone.
13. An aerosol provision system according to any preceding clause, further comprising an actuator.
14. An aerosol provision system according to clause 13, wherein the actuator is configured to be operated for allowing the sensor to operate.
15. An aerosol provision system according to clause 13 or 14, wherein the actuator is configured to be operated for allowing the operation to be performed.
16. An aerosol provision system according to any of clauses 13-15, wherein the control circuitry is configured to only perform the operation to the aerosol provision system in response to determining that both i) the sensor data satisfies the predetermined criterion; and ii) the actuator is being operated.
17. An aerosol provision system according to any of clauses 13-16, wherein the actuator comprises a touch sensor.
18. An aerosol provision system according to any of clauses 13-17, wherein the actuator comprises a capacitive sensor.
19. An aerosol provision system according to any of clauses 13-18, wherein the actuator is moveable between a first position in which the actuator is not operated, and a second position in which the actuator is operated.
20. An aerosol provision system according to any of clauses 13-19, wherein the actuator is moveable between a first position in which the sensor is not operated, and a second position in which the sensor is operated.
21. An aerosol provision system according to clause 19 or 20, wherein the actuator is biased towards the first position.
22. An aerosol provision system according to any preceding clause, wherein the aerosol provision system is configured to prevent the generation of aerosol from the aerosol provision system whilst the aerosol provision system is operating in the mode of operation.
23. An aerosol provision system according to any preceding clause, wherein the aerosol provision system further comprises an aerosol generator for generating the aerosol.
24. An aerosol provision system according to any clause 23, wherein the aerosol provision system is configured to prevent the operation of the aerosol generator whilst the aerosol provision system is operating in the mode of operation.
25. An aerosol provision system according to clause 23 or 24, wherein the operation comprises providing power to the aerosol generator from the aerosol provision system.
26. An aerosol provision system according to any preceding clause, wherein the operation comprises generating aerosol from the aerosol provision system.
27. An aerosol provision system according to any preceding clause, wherein the control circuitry is further configured to:
   receive second sensor data from the sensor;
   determine from the second sensor data whether the sensor data satisfies a second predetermined criterion; and
   perform a second operation of the aerosol provision system in response to determining that the second sensor data satisfies the second predetermined criterion;
   wherein the aerosol provision system is configured to operate in the mode of operation, prior to performing the operation of the aerosol provision system, for allowing the second predetermined criterion to be specified.
28. An aerosol provision system according to any preceding clause, wherein the aerosol provision system comprises an aerosol provision device and a consumable comprising aerosol-generating material for aerosolising, wherein the aerosol provision device is configured to releasably receive the consumable for allowing the aerosol-generating material to be aerosolised in use of the aerosol provision system.
29. An aerosol provision system according to clause 28, wherein the aerosol provision system is configured to operate in the mode of operation for sequentially allowing the first predetermined criterion, followed by the second predetermined criterion, to be specified.
30. An aerosol provision device, for use in an aerosol provision system for generating an aerosol comprising the aerosol provision device and a consumable comprising aerosol-generating material for aerosolising, wherein the aerosol provision device comprises a sensor, comprising a microphone, for detecting the voice of a user of the aerosol provision device, and wherein the aerosol provision device further comprises control circuitry which is configured to:
   receive sensor data from the sensor;
   determine from the sensor data whether the sensor data satisfies a predetermined criterion; and
   perform an operation of the aerosol provision device in response to determining that the sensor data satisfies the predetermined criterion;
   wherein the aerosol provision device is configured to operate in a mode of operation, prior to performing the operation of the aerosol provision device, for allowing the predetermined criterion to be specified.
31. A method of performing an operation in an aerosol provision system, the method comprising control circuitry from the aerosol provision system:
   operating the aerosol provision system in a mode of operation for allowing a predetermined criterion to be specified;
   receiving sensor data from a sensor, comprising a microphone, from the aerosol provision system, for detecting the voice of a user of the aerosol provision system;
   determining from the sensor data whether the sensor data satisfies the predetermined criterion; and
   performing an operation of the aerosol provision system in response to determining that the sensor data satisfies the predetermined criterion.
32. The method according to clause 31, wherein the method further comprises the control circuitry:
   performing the operation of the aerosol provision system only in response to determining that both i) the sensor data satisfies the predetermined criterion; and ii) an actuator from the aerosol provision system is being operated.
33. The method according to clause 32, wherein the method further comprises the control circuitry:
   stopping the operation as soon as the control circuitry determines that the actuator is no longer being operated.
34. The method according to any of clauses 31-33, wherein the method further comprises the control circuitry:
   operating the aerosol provision system in the mode of operation for allowing the operation to be specified.
35. The method according to any of clauses 31-34, wherein the method further comprises the control circuitry:
   preventing the generation of aerosol from the aerosol provision system whilst the aerosol provision system is operating in the mode of operation.
36. The method according to any of clauses 31-35, wherein the method further comprises the control circuitry:
   preventing the operation of an aerosol generator from the aerosol provision system whilst the aerosol provision system is operating in the mode of operation.

In order to address various issues and advance the art, this disclosure shows by way of illustration various embodiments in which the claimed invention(s) may be practiced. The advantages and features of the disclosure are of a representative sample of embodiments only, and are not exhaustive and/or exclusive. They are presented only to assist in understanding and to teach the claimed invention(s). It is to be understood that advantages, embodiments, examples, functions, features, structures, and/or other aspects of the disclosure are not to be considered limitations on the disclosure as defined by the claims or limitations on equivalents to the claims, and that other embodiments may be utilised and modifications may be made without departing from the scope of the claims. Various embodiments may suitably comprise, consist of, or consist essentially of, various combinations of the disclosed elements, components, features, parts, steps, means, etc. other than those specifically described herein, and it will thus be appreciated that features of the dependent claims may be combined with features of the independent claims in combinations other than those explicitly set out in the claims. The disclosure may include other inventions not presently claimed, but which may be claimed in future. In this respect as well, it may thus be appreciated that any features from any given independent claim or clause may comprise any combination of feature/features from any other herein recited clauses or claims.

As well, and as alluded to previously, it will also be appreciated that the present disclosure is not necessarily limited to only one predetermined criterion being paired with only a single operation of the aerosol provision system. Accordingly, it will be appreciated that the present disclosure is also applicable with aerosol provision systems which may employ any number of different operations of the aerosol provision system, wherein each operation is then pair/associated with a respective predetermined criterion (and potentially a commensurate predetermined utterance).

Equally, it will also be appreciated that the present disclosure is not necessarily limited to voice control from a single user. Thus in accordance with embodiments where the predetermined criterion comprises the sensor data being indicative of a voice pattern from a predetermined authorised user profile, in accordance with some embodiments, the predetermined criterion may equally comprise the sensor data being indicative of a voice pattern from one of a plurality of different predetermined authorised user profiles (e.g. in embodiments where a single aerosol provision system is shared by a several different users).

Yet further still, it will be appreciated in accordance with some embodiments that any predetermined criterion(s) used herein, which is paired with any given operation of the aerosol provision system 1, may comprise the sensor data being indicative of a predetermined utterance as having been spoken and also a voice pattern from a predetermined authorised user profile. In this way, for the operation of the aerosol provision system to be performed, the control circuitry 18 must determine that both the right utterance has been spoken, and that this utterance has been spoken by an authorised person/user. With respect to these latter embodiments, it may be therefore seen that this may then result in a particularly secure voice activated aerosol provision system 1 which is better resistant to unauthorised user operation.

Also, with respect to the aerosol provision system whilst it is operating in the mode of operation, it will be appreciated that in such instances, the aerosol provision system may in some embodiments be configured to prevent the generation of aerosol from the aerosol provision system whilst the aerosol provision system is operating in this mode of operation, and/or be configured to prevent the operation of any aerosol generator from the aerosol provision system whilst the aerosol provision system is operating in this mode of operation.

1. An aerosol provision system for generating an aerosol, wherein the aerosol provision system comprises:
a sensor, comprising a microphone, for detecting the voice of a user of the aerosol provision system;
an actuator; and
control circuitry which is configured to:
receive sensor data from the sensor;
determine from the sensor data whether the sensor data satisfies a predetermined criterion; and
perform an operation of the aerosol provision system only in response to determining that both i) the sensor data satisfies the predetermined criterion and ii) the actuator is being operated.

2. An aerosol provision system according to claim 1, wherein the control circuitry is configured to stop the operation as soon as the control circuitry determines that the actuator is no longer being operated.

3. An aerosol provision system according to claim 1, wherein the actuator comprises a touch sensor.

4. An aerosol provision system according to claim 1, wherein the actuator comprises a capacitive sensor.

5. An aerosol provision system according to claim 1, wherein the actuator is moveable between a first position in which the actuator is not operated, and a second position in which the actuator is operated.

6. An aerosol provision system according to claim 5, wherein the actuator is biased towards the first position.

7. An aerosol provision system according to claim 1, wherein the operation comprises providing power to an aerosol generator from the aerosol provision system.

8. (canceled)

9. (canceled)

10. An aerosol provision system according to claim 1, wherein the aerosol provision system is configured to operate in a mode of operation, prior to performing the operation of the aerosol provision system, for allowing the predetermined criterion to be specified.

11. An aerosol provision system according to claim 10, wherein the predetermined criterion comprises the sensor data being indicative of a predetermined utterance as having been spoken, wherein the predetermined utterance is indicative of a predetermined phrase as having been spoken.

12. (canceled)

13. An aerosol provision system according to claim 11, wherein the aerosol provision system is further configured to operate in the mode of operation for allowing the predetermined utterance to be specified, wherein the control circuitry is configured, whilst the aerosol provision system is operating in the mode of operation, to:
output a signal for instructing a user of the aerosol provision system to specify an utterance;
receive utterance data, from the sensor, containing data relating to the utterance;
determine the utterance from the utterance data; and
specify the predetermined utterance as being the determined utterance.

14. An aerosol provision system according to claim 10, wherein the predetermined criterion comprises the sensor data being indicative of a voice pattern from a predetermined authorised user profile.

15-18. (canceled)

19. An aerosol provision system according to claim 10, wherein the aerosol provision system is further configured, in the mode of operation, for allowing the operation of the aerosol provision system to be specified.

20. An aerosol provision system according to claim 19, wherein the aerosol provision system is configured for allowing the operation of the aerosol provision system to be specified via a user input device.

21. An aerosol provision system according to claim 19, wherein the aerosol provision system is configured for allowing the operation of the aerosol provision system to be specified via the microphone.

22. An aerosol provision system according to claim 10, wherein the aerosol provision system is configured to prevent the generation of aerosol from the aerosol provision system whilst the aerosol provision system is operating in the mode of operation.

23. An aerosol provision system according to claim 10, wherein the aerosol provision system further comprises an aerosol generator for generating the aerosol, wherein the aerosol provision system is configured to prevent the operation of the aerosol generator whilst the aerosol provision system is operating in the mode of operation.

24. (canceled)

25. An aerosol provision system according to claim 10, wherein the control circuitry is further configured to:
receive second sensor data from the sensor;
determine from the second sensor data whether the sensor data satisfies a second predetermined criterion; and
perform a second operation of the aerosol provision system in response to determining that the second sensor data satisfies the second predetermined criterion;
perform the second operation of the aerosol provision system only in response to determining that both i) the sensor data satisfies the second predetermined criterion and ii) the actuator is being operated.

26. An aerosol provision system according to claim 25, wherein the aerosol provision system is configured to operate in the mode of operation, prior to performing the operation of the aerosol provision system, for allowing the second predetermined criterion to be specified-and wherein the aerosol provision system is configured to operate in the mode of operation for sequentially allowing the first predetermined criterion, followed by the second predetermined criterion, to be specified.

27. (canceled)

28. (canceled)

29. An aerosol provision device, for use in an aerosol provision system for generating an aerosol comprising the aerosol provision device and a consumable comprising aerosol-generating material for aerosolising, wherein the aerosol provision device comprises:
a sensor, comprising a microphone, for detecting the voice of a user of the aerosol provision device;
an actuator; and
control circuitry which is configured to:
receive sensor data from the sensor;
determine from the sensor data whether the sensor data satisfies a predetermined criterion; and
perform an operation of the aerosol provision device only in response to determining that both i) the sensor data satisfies the predetermined criterion and ii) the actuator is being operated.

30. A method of performing an operation in an aerosol provision system, the method comprising control circuitry from the aerosol provision system:
receiving sensor data from a sensor, comprising a microphone, from the aerosol provision system, for detecting the voice of a user of the aerosol provision system;
determining from the sensor data whether the sensor data satisfies a predetermined criterion; and
performing an operation of the aerosol provision system only in response to determining that both i) the sensor data satisfies the predetermined criterion and ii) an actuator from the aerosol provision system is being operated.

31-40. (canceled)
